(19) United States
(12) Patent Application Publication
(10) Pub. No.: US 2026/0041619 A1
(43) Pub. Date: Feb. 12, 2026

(54) TRANSPARENT COSMETIC AND PERSONAL CARE COMPOSITIONS

(71) Applicant: Conopco, Inc., d/b/a UNILEVER, Englewood Cliffs, NJ (US)

(72) Inventors: Souad ASSIGHAOU, Irby (GB); Kelvin Brian DICKINSON, Bebington, Wirral, Merseyside (GB); Leanne Chelsea JENKINS, Burscough (GB); Hailey KELSO, Chester, Cheshire (GB)

(21) Appl. No.: 18/995,656

(22) PCT Filed: Aug. 1, 2023
(86) PCT No.: PCT/EP2023/071324

§ 371 Date: Jan. 16, 2025

(30) Foreign Application Priority Data
Aug. 15, 2022 (EP) 22190399.0

(51) Int. Cl. A61K 8/41; A61K 8/49; A61K 8/73; A61Q 5/12

(52) U.S. Cl. CPC A61K 8/41; A61K 8/4926; A61K 8/731; A61Q 5/12; A61K 2800/262; A61K 2800/5922

(57) ABSTRACT

An aqueous conditioner composition comprising: i) 0.1 to 2 wt % of a cationic conditioning polymer: ii) 0.1 to 5 wt % of an ethoxylated anionic surfactant comprising ethylene oxide groups, with a degree of ethoxylation of from 3 to 15; and iii) 0.001 to 2 wt % of a piroctone compound: wherein the composition has a transparency such that the turbidity is lower than 1 cm−1, as measured using a UV/vis spectrophotometer and applying the equation Turbidity=(2.3×A/L), where A is the absorbance measured from the sample at 750 nm and L is the path length; and wherein the composition has a maximum foam height of 10 ml, as measured at 25° C. and atmospheric pressure, by diluting 1 g composition with 9 g water in a 100 ml graduated cylinder with an internal diameter of 29 mm, and recording a starting volume (V1); the cylinder then being stoppered and vigorously shaken vertically for 10 seconds, followed by resting for 60 seconds after which measuring the height of the foam to the nearest 5 ml graduation; and subtracting the starting volume (V1) from this value; and wherein the composition is free from anionic, zwitterionic and amphoteric surfactants other than those defined in ii), such that the foam height of the composition does not exceed the maximum foam height.

### TECHNICAL FIELD

This invention relates to a transparent anti-dandruff conditioner composition, and to a method of treating hair with said composition.

### BACKGROUND

Cosmetic and personal care compositions are often required to have multiple visual and functional attributes. For example, shampoo and hair conditioner compositions are generally required to have properties in addition to their ability to clean and/or condition hair in order to be appealing to the consumer.

Clear, transparent compositions are often desirable visually, but difficult to formulate. Additional benefit agents, such as antidandruff agents can cause undesirable effects such as clouding and discolouration.

Hair conditioners are typically used after shampooing and are required to deliver high levels of conditioning feel (slippery, smooth, lubricity, detangling) to wet hair. Foaming during use is strongly disliked by consumers. Most conditioners are effective because they contain a dispersed lamellar gel phase that is highly efficient at detangling. However, these are opaque because of the dispersed phase. Attempts have been made in the past to make the dispersed lamellar gel phase fragments small enough to make the composition transparent but processing and stability problems occur. Other known transparent conditioners are mainly polymer based and less effective at delivering conditioning benefits.

In contrast, shampoos are formulated to foam during use and as such typically comprise high levels of foaming cleansing surfactants, which are typically anionic, zwitterionic/amphoteric and nonionic in nature. The consumer associates foaming with cleansing performance. Shampoos deliver a much lower level of conditioning benefit, particularly in the wet, to hair. Thus, consumers use both shampoo and conditioner in a typical wash regime, traditionally using the shampoo first, followed by the conditioner.

Antidandruff shampoos and conditioners can contain piroctone compounds, which are deposited onto hair and scalp during use. However, the piroctone actives that are deposited do not remain there, due to deterging by the surfactants present in the formulations. The piroctone is, therefore, mostly washed away.

It is known to increase the amount of piroctone compound in order to achieve increased antidandruff benefit. For example:

WO2021/144267A1 discloses that personal cleansing compositions, such as shampoos, that comprise i) a piroctone compound, ii) a cleansing surfactant that comprises amphoteric surfactant and ethoxylated alkyl sulphate in a given ratio and iii) a cationic polymer comprising polyquaternium-6 provide improved deposition of piroctone compounds onto the surface of scalp and/or hair during washing.

JP H1135424A provides a specific guanidine derivative in shampoo and anti-dandruff shampoo, to obtain a hair cosmetic and sensory conditioning benefits.

EP2944852A1 discloses the use of a booster selected from alcohols having 2 to 5 hydroxy groups and plant extracts to enhance of anti-dandruff or preservative activity of piroctone olamine in shampoos.

Despite the prior art, there remains a need for transparent conditioner compositions that deposit piroctone compounds onto hair and scalp more efficiently with reduced deterging during the wash process.

We have now found that a transparent conditioner comprising a piroctone antidandruff agent, can be provided by use of a cationic polymer in combination with an ethoxylated anionic surfactant that comprises a high number of ethylene oxide groups, to give excellent visual attributes without compromising on conditioning performance and displaying minimal foam. Surprisingly, this transparent conditioner provides a dramatic increase in deposition efficiency of piroctone compounds, thus providing excellent antidandruff benefit with a lower amount of piroctone.

### DEFINITION OF THE INVENTION

According to the present invention, in a first aspect, there is provided an aqueous conditioner composition comprising:

An aqueous conditioner composition comprising:
i) 0.1 to 2 wt % of a cationic conditioning polymer;
ii) 0.1 to 5 wt % of an ethoxylated anionic surfactant, with a degree of ethoxylation of from 3 to 15; and
iii) 0.001 to 2 wt % preferably 0.01 to 1 wt %, more preferably 0.05 to 0.5 wt % of a piroctone compound;
wherein the composition has a transparency such that the turbidity is lower than 1 cm⁻¹, as measured using a UV/vis spectrophotometer and applying the equation Turbidity=(2.3×A/L), where A is the absorbance measured from the sample at 750 nm and L is the path length;

wherein the composition has a maximum foam height of 10 ml, as measured at 25° C. and atmospheric pressure, by diluting 1 g composition with 9 g water in a 100 ml graduated cylinder with an internal diameter of 29 mm, and recording a starting volume (V1); the cylinder then being stoppered and vigorously shaken vertically for 10 seconds, followed by resting for 60 seconds after which measuring the height of the foam to the nearest 5 ml graduation; and subtracting the starting volume (V1) from this value; and wherein the composition is free from anionic, zwitterionic and amphoteric surfactants other than those defined in ii), such that the foam height of the composition does not exceed the maximum foam height.

In a second aspect, the invention provides a non-therapeutic method of treating hair or scalp, comprising applying to the hair or scalp a composition in accordance with the first aspect of the invention.

### GENERAL DESCRIPTION OF THE INVENTION

The antidandruff compositions of the invention are transparent and low foaming.

The level of foaming produced by the compositions of the invention can be measured using any suitable method. A preferred method is a Cylinder Shake Test, as follows.

The cylinder shake test is preferably carried out under ambient conditions (25° C. and atmospheric pressure).

The test product (1 g) is diluted with water (9 g) and added to a 100 ml graduated cylinder. The starting volume (V1) is recorded. The cylinder is stoppered and then vigorously shaken vertically for 10 seconds. After a further 60 seconds rest, the height of the foam is measured visually to the nearest 5 ml graduation. The starting volume (V1) of solution is subtracted from this value, the result of which indicates the amount of air entrained in the foam, or “foam height”.

A suitable 100 ml graduated glass cylinder is one manufactured by Duran, supplied by VWR and with an internal diameter of 29 mm.

By this method, the maximum height of foam produced by compositions of the invention is 10 ml, preferably 7 ml, more preferably 5 ml.

A suitable method of assessing transparency is to measure turbidity. UV-vis spectrometry may be used to determine the turbidity of the formulation. An example of a suitable spectrophotomer is a Jasco V-650 spectrophotometer.

Translucency (or turbidity) in a liquid product is due to suspended or colloidal particles that cause light to be scattered rather than transmitted in straight lines through the sample.

Turbidity may be calculated using the equation, where turbidity is 2.3 multiplied by absorbance divided by the path length of the sample, i.e. Turbidity=(2.3×A/L); where A is the absorbance measured from the sample at 750 nm and L is the path length. Preferably a path length of 1.0 cm is used.

A composition is said to be transparent when the turbidity is lower than 1 cm⁻¹, preferably lower than 0.5 cm⁻¹, more preferably lower than 0.4 cm⁻¹, even more preferably lower than 0.25 cm⁻¹ most preferably lower than 0.1 cm⁻¹, as measured using a UV/visible spectrophotometer and applying the equation turbidity=(2.3*A/L), where A is the absorbance measured from the sample at 750 nm and L is the path length.

### The Cationic Conditioning Polymer

The compositions of the invention comprise at least one cationic conditioning polymer.

The cationic conditioning polymer is present in an amount of 0.1 to 2 wt %, preferably 0.1 to 1 wt %, by total weight of the composition.

Preferably, the polymer has a polysaccharide backbone, wherein the polysaccharide comprises cationic modification. Preferably, the cationic modification comprises an amino group, for example a quaternary ammonium group.

Preferably the polysaccharide backbone is cellulosic. Most preferably the cellulosic backbone is hydroxy ethyl cellulose.

A preferred polymer is commercially available as Polyquaternium-10 (PQ10) for example UCare™ polymer JR-30M from Dow.

### The Ethoxylated Anionic Surfactant

The compositions of the invention comprise an ethoxylated anionic surfactant. The soluble ethoxylated anionic surfactant comprises ethylene oxide (EO) groups, with a degree of ethoxylation (n) of from 3 to 15, preferably from 5 to 15.

The ethoxylated anionic surfactant is preferably present in an amount of 0.1 to 5 wt %, preferably 0.1 to 2 wt %, by weight of the total composition.

Preferably the ethoxylated anionic surfactant is linear.

The anion is preferably a phosphate or a sulphate group.

Examples of preferred ethoxylated anionic surfactants are Oleth-10-Phosphate and PPG-5-Ceteth-10 Phosphate.

These are available from Croda as Crodafos™ 010A, and Crodafos™ SG respectively.

The level of ethoxylation may be measured by any suitable method. One method is by equilibrium headspace analysis,

### The Piroctone Compound

The piroctone compound useful in the present invention typically contains the structure defined by formula (A):

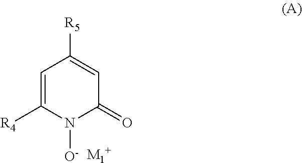

wherein R₄ is selected from C1-C17 hydrocarbon radicals, R₅ is selected from C1-4 alkyl, C2-4 alkenyl or alkynyl, hydrogen, phenyl or benzyl, and M₁ is selected from hydrogen, monoethanolamine (MEA), diethanolamine (DEA), or triethanolamine (TEA). Preferred R₄ group is (CH₃)₃CCH₂CH(CH₃)CH₂— and preferred R₅ is a methyl. More preferably, R₄ is (CH₃)₃CCH₂CH(CH₃)CH₂—, R₅ is a methyl and M₁ is a hydrogen or MEA. Most preferably, R₄ is (CH₃)₃CCH₂CH(CH₃)CH₂—, R₅ is a methyl and M₁ is monoethanolamine.

The piroctone compound for use in the present invention is preferably selected from piroctone acid, primary olamine salts of piroctone acid, secondary olamine salts of piroctone acid and tertiary olamine salts of piroctone acid, and mixtures thereof, more preferably selected from piroctone acid, primary olamine salts of piroctone acid and mixtures thereof.

A preferred example of a primary olamine salt of piroctone acid is primary olamine salts of piroctone acid is piroctone olamine, which is available as Octopirox®. An example of a suitable secondary olamine salt of piroctone acid is the diethanolamine salt; an example of a suitable teritiary salt of piroctone acid is the triethanolamine salt.

Piroctone olamine is particularly preferred.

The typical level of the piroctone compound is from 0.001 to 2 wt % preferably 0.01 to 1 wt %, more preferably 0.05 to 0.5 wt %, most preferably from 0.05 to 0.3 wt %.

In compositions and method according to the invention, some or all of the piroctone compound is in dissolved form such that the composition is transparent. Some of the piroctone compound may not be in dissolved form. The amount of undissolved AD agent is low enough to maintain the transparency of the composition.

### Solvent

A solvent is preferably present in order to increase the solubility of the piroctone compound in the composition. The solvent enables more piroctone compound to be incorporated into the composition whilst maintaining transparency. The solvent is capable of dissolving the piroctone compound at ambient temperature. Typically, ambient temperature is from 20 to 30 degrees.

Preferred solvents are alcohols, preferably selected from propylene glycol (PG), dipropylene glycol (DPG) and mixtures thereof.

The solvent is preferably present in an amount of 0.025 to 5 wt %, more preferably 0.05 wt % to 3 wt %, even more preferably 0.5 wt % to 2 wt %, by total weight of the composition.

### Cationic Surfactant

The composition preferably comprises a cationic surfactant. The presence of a cationic surfactant aids transparency. This is particularly useful if other components, for example perfume, are added.

Preferred cationic surfactants may be selected from quaternary ammonium surfactants and tertiary ammonium surfactants.

Preferred cationic surfactants include coco-amine ethoxylates, for example Ethomeen™ C/25 or C/15, available from Nouryon. Also, PEG-15 Cocamine and PEG-2 Cocamine and cetyltrimethylammonium chloride cetyltrimethylammonium chloride (CTAC).

Compositions of the invention comprise cationic surfactants preferably comprising amino or quaternary ammonium hydrophilic moieties which are positively charged when dissolved in an aqueous composition.

Examples of suitable cationic surfactants correspond to the following general formula:

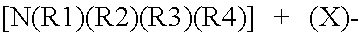

in which R1, R2, R3, and R4 are each independently selected from
(a) an aliphatic group of from 1 to 22 carbon atoms, or
(b) hydrogen or a polyoxyalkylene, hydroxyalkyl, aromatic, alkoxy, alkylamido, hydroxyalkyl, aryl, alkylaryl group having up to 22 carbon atoms; and
   X is a salt-forming anion such as those selected from halide, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated.

Specific examples of such quaternary ammonium cationic surfactants of the above general formula are PEG-15 Cocamine and PEG-2 Cocamine, cetyltrimethylammonium chloride (CTAC) and salts of these, where the chloride is replaced by other halide (e.g., bromide), acetate, citrate, lactate, glycolate, phosphate nitrate, sulphate, or alkylsulphate.

In a preferred class of cationic surfactant of the above general formula, R1 is a C2 to C22 saturated or unsaturated, preferably saturated, alkyl chain and R², R³ and R4 are each independently selected from CH₃ and (CH₂CH₂O)nH, preferably (CH₂CH₂O)nH.

The level of cationic surfactant is preferably from 0.05 to 5, preferably 0.1 to 2 wt. % of the total composition. Some cationic surfactants may produce foam in the compositions of the invention.

Where present, the amount of cationic surfactant is such that the maximum foam height, defined and as determined herein, is not exceeded.

A further acid used may be used to protonate the amine. Suitable acids include hydrochloric acid, citric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, lactic acid and mixtures thereof.

Mixtures of any of the above-described cationic surfactants may also be suitable.

### Rheology Modifier

The compositions of the invention preferably comprise a rheology modifier to provide improved spreading of the composition on the hair. This typically thickens the composition.

Where present, preferably, the rheology modifier is present in an amount of from 0.2 to 2 wt %, most preferably from 0.5 to 1.5 wt %.

Preferably, the rheology modifier is a polysaccharide, preferably derived from cellulose.

Preferably, the rheology modifier is non-ionic.

Preferably, the structurant has a molecular weight ranging from 500 kDa to 2 MDa.

Examples of suitable rheology modifier include Hydroxy Ethyl Cellulose (HEC) available, for example, under the tradename Natrosol, in a range available from Ashland. Another suitable example is Amaze XT, available from Nouryon.

Preferred rheology modifiers include hydroxy ethyl cellulose and hydroxy propyl methyl cellulose. The most preferred structurant is Hydroxy Ethyl Cellulose.

### Transparency Aid

The compositions may comprise a transparency aid to improve transparency. The transparency aid is different from and not intended to be the same as any solvent as described above for the piroctone compound.

A transparency aid is particularly useful to aid solubilisation of components such as perfumes.

Preferred transparency aids are non-ionic in character. They may be selected from nonionic surfactants and nonionic emulsifiers.

Preferred transparency aids are Polysorbate 20, for example Tween™, available from Croda and Laureth-7, for example Marlipal 24/70 from Sasol.

Some transparency aids may produce foam in the compositions of the invention. Where present, the transparency aid is present in such an amount that it does not cause the foam height to exceed the maximum defined herein. As such, the transparency aid, where present, is preferably present in an amount of from 0.1 to 5 wt %, more preferably from 0.2 to 2 wt %, most preferably from 0.25 to 1.5 wt %, by weight of the total composition.

The compositions of the invention comprise water. The compositions suitably comprise water in amount of from 60 to 98 wt % preferably from 80 to 97 wt %, most preferably from 90 to 97 wt %, by weight of the total composition.

The compositions of the invention are preferably free from dispersed phases that affect the transparency (ie are not dissolved), for example silicone emulsion.

Such dispersed phases have dispersed droplets of water-insoluble material. For example conditioning agents. Examples of water-insoluble conditioning agents include non-silicone conditioning agents comprising non-silicone oily or fatty materials such as hydrocarbon oils, fatty esters and mixtures thereof. Preferably, the water-insoluble conditioning agent is emulsified silicone oil.

In the context of the invention, by free from dispersed phases, is meant having less 0.1 weight %, preferably less than 0.05 weight %, more preferably less than 0.001 weight %, yet preferably less than 0.0001 weight %, and most preferably 0 weight % of dispersed phases by weight of the total composition.

The pH of the compositions is preferably from 3 to 7, more preferably 3.5 to 6.5, most preferably from 5 to 6.

The composition is free from anionic, zwitterionic and amphoteric surfactants, other than those defined in ii), such that the foam height of the composition does not exceed the maximum foam height.

In the context of this invention, by free from anionic, zwitterionic and amphoteric surfactants, other than those defined in ii) means preferably the level of these foam-producing anionic, zwitterionic and amphoteric surfactants is less than 1.0 weight %, more preferably less than 0.5 weight %, more preferably less than 0.1 weight %, still more preferably less than 0.001 weight %, and most preferably 0 weight % of surfactant by weight of the total composition. It is possible, for example, that such surfactants may be present as “carry over” in raw materials used in the compositions of the inventions. The amount of the surfactants is such that the foam height of the composition does not exceed the maximum foam height.

In particular, these anionic, zwitterionic and amphoteric surfactants preferably include the following types of surfactant:
Surfactants with alkyl chains of less that 16 C≡C atoms;
Polyoxyethylene alkyl ether sulphate salts having an average mole number of added ethylene oxide in the range of from 1 to 5, preferably 1 to 3;
Betaines (with alkyl chain length of less than 16). For example, oleyl betaine, caprylamidopropyl betaine, lauramidopropyl betaine, isostearylamidopropyl betaine, and cocoamidopropyl betaine;
Amine oxides, for example lauramine oxide and cocamine oxide;
Cocamide monoethanolamine (CMEA);
Amphoacetates;
Taurates, isothionates, glycinates and suphonates, for example alpha olefin sulphonate.

### Further Ingredients

The composition according to the invention may comprise any of a number of ingredients which are common to hair compositions.

Other ingredients may include deposition polymers, preservatives, colouring agents, polyols such as glycerine and polypropylene glycol, chelating agents such as EDTA, antioxidants such as vitamin E acetate, fragrances, antimicrobials and pH adjusters for example acids, preferably organic acids. Each of these ingredients will be present in an amount effective to accomplish its purpose.

Preferably, the further ingredients include perfumes, preservatives and antimicrobials.

Mixtures of any of the above active ingredients may also be used.

Generally, such ingredients may be included individually at a level of up to 5 wt %, preferably 2%, most preferably up to 1%, by weight of the total composition.

Embodiments of the invention are given in the following examples, in which all percentages are quoted by weight based on total weight unless otherwise stated.

### EXAMPLES

The invention will now be illustrated by the following non-limiting examples. In the examples and throughout this specification, all percentages are by weight based on total composition unless indicated otherwise.

### Example 1—Preparation of Compositions 1˜4 in Accordance with the Invention and Comparative Compositions a & B

The following compositions were prepared, where 1-4 are in accordance with the invention and A and B are comparatives:

Conditioner composition 1 comprises a cationic conditioning polymer, an ethoxylated anionic surfactant comprising 10 EO groups and piroctone olamine at 0.1 wt %.

Conditioner Compositions 2-4 additionally comprise cationic surfactant, rheology modifier, solvent (propylene glycol or dipropylene glycol) and various amounts of piroctone olamine.

Comparative Conditioner Composition A is representative of a typical prior art shampoo composition and comprises sodium lauryl ether phosphate (SLES) 1EO and cocoamidopropyl betaine and 0.5 wt % piroctone olamine.

Comparative Conditioner Composition B is the same as Composition 1, but without piroctone olamine.

The following commercially available shampoos were used in the tests below:

Control 1: A commercially available antidandruff shampoo, “Clear Antidandruff Nourishing Shampoo Anti Hair Fall Womens” was used as a control in piroctone olamine deposition tests. This product contains 0.50% Octopirox.

Control 2: A commercially available antidandruff shampoo, “Head and Shoulders 2 in 1 Men Total Care with sea minerals” was used as a control in piroctone olamine deposition and foam generation tests. This product contains at least 0.50% Octopirox.

The compositions of Compositions 1-4 and A & B are given in Table 1 below:

TABLE 1
Compositions of Compositions 1-4, in accordance with
the invention and Comparative Compositions A and B

| | Amount (% wt) | | | | | |
|---|---|---|---|---|---|---|
| Ingredients INCI | A | B | 1 | 2 | 3 | 4 |
| PEG-15 Cocamine | — | | — | 1 | 1 | 1 |
| Hydroxyethylcellulose | — | | — | 1 | 1 | 1 |
| Polyquaternium-10 | — | 0.5 | 0.5 | 0.5 | 0.5 | 0.5 |
| Polysorbate 20 | — | — | — | 1 | 1 | 1 |
| Oleth-10-Phosphate | — | 1 | 1 | 1 | 1 | 1 |
| Sodium laureth | 14 | — | — | — | — | — |
| sulfate | | | | | | |
| Cocamidopropyl | 2 | — | — | — | — | — |
| betaine | | | | | | |
| Citric Acid | To pH 5 | To pH 5 | To pH 5 | To pH 5 | To pH 5 | To pH 5 |
| Perfume | 0.8 | 0.8 | 0.8 | 0.8 | 0.8 | 0.8 |
| Piroctone olamine | 0.5 | — | 0.10 | 0.10 | 0.15 | 0.25 |
| (Octopirox ex | | | | | | |
| Clariant) | | | | | | |
| Propylene glycol | — | — | — | — | 1.20 | — |
| Dipropylene glycol | — | — | — | — | — | 2 |
| Water and minors | up to | up to | up to | up to | up to | up to |
| (preservatives) | 100% | 100% | 100% | 100% | 100% | 100% |

The compositions in Table 1 were prepared as follows:
1. Water (300 ml) was provided in a first vessel at 25° C.
2. Where present, hydroxyethylcellulose was added to the water, with stirring for 2 minutes.
3. The cationic conditioning polymer (Polyquaternium-10) was added and the mixture stirred for a further 2 minutes.
4. Where present, cationic surfactant (PEG-15 cocamine or PEG-2 Cocamine) was added and the mixture stirred for a further 2 minutes.
5. In a second vessel, anionic surfactant was added along with any minor ingredients to water (35 ml) at 70° C. and gently stirred until dissolved.
6. The resulting hot solution was then added to the mixture in the first vessel, which was heated to 40° C. with stirring at 100 rpm for 30 minutes before being cooled to room temperature.
7. Any remaining ingredients (fragrance, polysorbate 20, citric acid, piroctone olamine (added as a slurry with water)) were added with continued stirring at 100 rpm.

### Example 2—Deposition of Piroctone Olamine onto Hair Treated with Conditioner Compositions 1˜4 in Accordance with the Invention, Comparative Compositions a & B and Shampoo Controls 1 & 2

### Treatment of Hair

Dark brown European virgin hair switches (2.5 g, 6 inch) were used in the following tests and procedures.

Hair switches were twice pre-washed with a base shampoo composition to remove any surface contamination before starting any treatment. Each switch was wetted then treated with 0.1 g shampoo per g of hair and lathered for 30 s before being rinsed in warm running water for 30 s. The hair was combed and excess water gently squeezed out.

For the shampoo controls, (Controls 1 and 2) and comparative composition A, 0.1 g shampoo per g of hair was applied to the hair and lathered for 30 s before being rinsed in warm running water for 30 s. A second application and rinse was performed in the same way. The hair was combed and excess water gently squeezed out.

For each conditioner formulation (Compositions 1˜4 and Composition B), 0.2 g of conditioner formulation per 1 g of hair was applied evenly to the washed switches (3 switches per composition) and massaged into the hair before being rinsed for 1 minute. Excess water was then gently removed and the switches dried at 50° C. in a drying cabinet.

Note that the shampoo formulations were applied twice to hair at 0.1 g product per gram of hair, with a rinse stage after each application, whilst the conditioner formulations were applied once to hair at 0.2 g per gram of hair. These applications are typical of consumer behaviour.

### Extraction of Switches for Piroctone Olamine Deposition Analysis:

Each dried switch was placed into a suitable jar and ca. 10 ml of Ethanol Absolute was added. The jar was then sealed and placed onto a roller bed for 1 hour, after which a syringe was used to remove the extraction liquor.

Deposition of Octopirox onto Hair

The amount of Octopirox deposited onto the hair was measured by HPLC. The results are given in Table 2 below.

TABLE 2
Deposition of piroctone olamine (Octopirox) onto hair treated
with Conditioner Compositions 1-4 in accordance with the invention,
Comparative Compositions A & B and Shampoo Controls 1 & 2

| Example | Mean (PPM) | SD | Std Error |
|---|---|---|---|
| Control 1 (0.50% OCT) | 11.66749 | 2.082673 | 1.202432 |
| Control 2 (0.50% OCT) | 10.9124 | 1.26773 | 0.731924 |
| Composition A (0.50% OCT) | 8.001325 | 1.351947 | 0.780547 |
| Comparative B (0% OCT) | −0.14572 | 0 | 0 |
| Composition 1 (0.10% OCT) | 5.368438 | 0.811366 | 0.468442 |
| Composition 2 (0.10% OCT) | 4.682895 | 2.885671 | 1.666043 |
| Composition 3 (0.15% OCT) | 7.514489 | 1.389112 | 0.802004 |
| Composition 4 (0.25% OCT) | 6.977976 | 1.07468 | 0.620467 |

### Example 3—Efficiency of Deposition of Piroctone Olamine (Octopirox) onto Hair Treated With Conditioner Compositions 1˜4 in Accordance With the Invention, Comparative Composition A & B and Shampoo Controls 1 & 2

### Efficiency of Deposition

### Theoretical Maximum Deposition

The theoretical maximum deposition of Octopirox onto hair was calculated by taking the amount of Octopirox in the composition (wt %) and multiplying by the dosage applied to the hair (0.2 g) and expressing in ppm.

The measured deposition (ppm) of Octopirox on the hair, following treatment as described above, can be divided by the theoretical maximum to give a deposition efficiency. This reflects the proportion of Octopirox that is retained on the hair.

The deposition efficiency is given in Table 3.

TABLE 3
Theoretical maximum deposition of, measured deposition
of and calculated Deposition Efficiency of piroctone
olamine (Octopirox) onto hair treated with Conditioner
Compositions 1-4 in accordance with the invention, Comparative
Compositions A & B and Shampoo Controls 1 & 2

| | | Measured | |
|---|---|---|---|
| | | deposition | |
| | Theoretical | following | Deposition |
| | maximum | rinsing | Efficiency |
| Example | (ppm) | (ppm) | (%) |
| Control 1 (0.50% OCT) | 1000 | 11.66749 | 1.16 |
| Control 2 (0.50% OCT) | 1000 | 10.9124 | 1.09 |
| Composition A (0.50% OCT) | 1000 | 8.001325 | 0.80 |
| Comparative B (0% OCT) | 0 | −0.14572 | — |
| Composition 1 (0.10% OCT) | 200 | 5.368438 | 2.68 |
| Composition 2 (0.10% OCT) | 200 | 4.682895 | 2.34 |
| Composition 3 (0.15% OCT) | 300 | 7.514489 | 2.50 |
| Composition 4 (0.25% OCT) | 500 | 6.977976 | 1.40 |

It can be seen that the deposition of Octopirox from these rinse-off products is an inefficient process, with the majority of the antidandruff benefit agent present being removed during the rinsing step.

The Control shampoos, 1 and 2, and Comparative Example A all deliver Octopirox with a deposition efficiency in the range of from 0.80 to 1.16%.

Compositions 1-4, according to the invention, deliver Octopirox with a markedly higher deposition efficiency of 1.40 to 2.68%.

### Example 4: Foaming Properties of Compositions 1 & 2, in Accordance with the Invention, and Comparative Composition A

Consumers do not accept foaming from a hair conditioner during use. Foam is strongly associated with cleansing and not with caring.

The foaming levels of conditioner compositions 1, 2 and A were evaluated.

### Cylinder Shake Foam Test

The level of foaming produced by the compositions of the invention was measured using the following method at ambient temperature (25° C.) and atmospheric pressure.

1 g of the test product was diluted with 9 g of water and added to a 100 ml graduated glass cylinder, manufactured by Duran, supplied by VWR, and with an internal diameter of 29 mm. The starting volume (V1) was recorded. The cylinder was stoppered and then vigorously shaken vertically for 10 seconds. After a further 60 seconds rest, the height of the foam was measured visually to the nearest 5 ml graduation. The starting volume of solution (V1=10 ml) was subtracted from this value to calculate the amount of air entrained in the foam.

The results are shown in Table 4.

TABLE 4
Foam height for compositions 1, 2 and A

Height of Foam; n = 5

| | | Standard |
|---|---|---|
| | Average/ | Deviation/ |
| Composition Description | ml | ml |
| 1 | 5 | 0 |
| 2 | 5 | 0 |
| A | 85 | 0 |

It will be seen that the compositions of the invention exhibit very low levels of foam.

### Example 5: Transparencies of Compositions 1-4, in Accordance with the Invention, and Comparative Composition B

The transparencies of conditioner compositions 1˜4 and B were evaluated by measuring the turbidity. The conditioner formulation is said to be transparent when the turbidity is lower than 1.0 cm⁻¹.

The transparency of each formulation was measured using a Jasco V-650 spectrophotometer. Absorbance at 750 nm was converted into a turbidity value according to the equation: Turbidity=(2.3× Absorbance)/path length, where A is the absorbance measured from the sample at 750 nm and the path length, which was 1.0 cm.

Turbidities of Compositions 1˜4 and B are given in Table 5 below.

TABLE 5
Turbidities of Compositions 1-4 and B

| Example | Turbidity (cm⁻¹) | Amount of Octopirox (wt %) |
|---|---|---|
| 1 | 0.00 | 0.1 |
| 2 | 0.29 | 0.1 + transparency aid |
| 3 | 0.01 | 0.15 + transparency aid + PG |
| 4 | 0.04 | 0.25 + transparency aid + DPG |
| B | 0.01 | 0 |

A transparent sample has a turbidity below 1.0 cm⁻¹.
At the lowest level (0.1 wt %) of Octopirox (Example 1) transparency is achieved without transparency aid or solvent.
Example 2 shows that transparency can be maintained, even when a rheology modifier and cationic surfactant are introduced, by the addition of a transparency aid.
Example 3 & 4 show that excellent transparency can be achieved at higher Octopirox levels with addition of solvent, in accordance with the invention.

### Example 6: Conditioning Properties (Level of Friction on Treated Hair) of Compositions 2, 3 and a Commercially Available Transparent Conditioner

The level of friction of hair may be used as an indication of the level of lubricity imparted to hair by a conditioning treatment.

Coefficient of friction is a dimensionless number that is defined as the ratio between friction force and normal force.

Hair (5 g hair switch) was first wetted with water and treated with 1 g of composition (2 and 3) before being diluted with a further 7 ml of water.

Coefficients of friction (CoF) were then determined by stroking a hair switch with a real finger on a bespoke instrumented force plate which used 6 force transducers to measure forces and torques in three dimensions.

A commercially available transparent conditioner, “L'Oreal Fibrology Transparent Conditioner”, was also used as a comparison.

The results are given in Table 6.

TABLE 6
Coefficient of friction of hair treated with compositions 2
and 3 and a commercially available transparent conditioner

| Composition | CoF after 7 ml water added |
|---|---|
| 2 | 0.32 |
| 3 | 0.32 |
| L'Oreal Fibrology | 0.41 |
| Transparent Conditioner | |

Compositions 2 and 3 provided superior lower friction.

1. An aqueous conditioner composition comprising:
i) 0.1 to 2 wt % of a cationic conditioning polymer;
ii) 0.1 to 5 wt % of an ethoxylated anionic surfactant comprising ethylene oxide groups, with a degree of ethoxylation of from 3 to 15; and
iii) 0.001 to 2 wt % of a piroctone compound:
wherein the composition has a transparency such that the turbidity is lower than 1 cm⁻¹, as measured using a UV/vis spectrophotometer and applying the equation Turbidity=(2.3× A/L), where A is the absorbance measured from the sample at 750 nm and L is the path length;
wherein the composition has a maximum foam height of 10 ml, as measured at 25° C. and atmospheric pressure, by diluting 1 g composition with 9 g water in a 100 ml graduated cylinder with an internal diameter of 29 mm, and recording a starting volume (V1); the cylinder then being stoppered and vigorously shaken vertically for 10 seconds, followed by resting for 60 seconds after which measuring the height of the foam to the nearest 5 ml graduation; and subtracting the starting volume (V1) from this value; and
wherein the composition is free from anionic, zwitterionic and amphoteric surfactants other than those defined in ii), such that the foam height of the composition does not exceed the maximum foam height.

2. The composition as claimed in claim 1, further comprising 0.025 to 5 wt % of a solvent that dissolves the piroctone compound at ambient temperature.

3. The composition as claimed in claim 2, wherein the solvent is selected from propylene glycol, dipropylene glycol and mixtures thereof.

4. The composition as claimed in claim 1, wherein the piroctone compound is selected from the group consisting of piroctone acid, primary olamine salts of piroctone acid, secondary olamine salts of piroctone acid, tertiary olamine salts of piroctone acid and mixtures thereof.

5. The composition as claimed in claim 4, wherein the piroctone compound is piroctone olamine.

6. The composition as claimed in claim 1, further comprising 0.05 to 5 wt % of a cationic surfactant.

7. The composition as claimed in claim 6, wherein the cationic surfactant is selected from a quaternary ammonium surfactant and a tertiary ammonium surfactant.

8. The composition as claimed in claim 1, wherein the cationic conditioning polymer has a polysaccharide backbone wherein the polysaccharide comprises cationic modification.

9. The composition as claimed in claim 8, wherein the cationic modification comprises an amino group.

10. The composition as claimed in claim 1, wherein the ethoxylated anionic surfactant has a degree of ethoxylation, n, of from 5 to 15.

11. The composition as claimed in claim 1, further comprising a rheology modifier in an amount of from 0.2 to 2 wt %.

12. The composition as claimed in claim 1, further comprising a transparency aid.

13. The composition as claimed in claim 12, wherein the transparency aid is present in an amount of from 0.1 to 5 wt % such that the maximum foam height, defined in claim 1, is not exceeded.

14. The composition as claimed in claim 1, which is free from dispersed phases that affect the transparency.

15. A method of treating hair or scalp, comprising applying the composition of claim 1 to the hair or scalp.

16. The composition as claimed in claim 12, wherein the transparency aid is a nonionic surfactant or a nonionic emulsifier.
